(19) United States
(12) Patent Application Publication
(10) Pub. No.: US 2015/0050210 A1
(43) Pub. Date: Feb. 19, 2015

(54) PHARMACEUTICAL FORMULATION COMPRISING BENDAMUSTINE

(71) Applicant: Salmon Pharma GmbH, Basel (CH)

(72) Inventors: Akif Emre Tuereli, Saarlouis (DE); Bernd Baumstuemmler, Saarlouis (DE); Richard Ammer, Iserlohn (DE)

(21) Appl. No.: 14/387,426

(22) PCT Filed: Mar. 25, 2013
(86) PCT No.: PCT/EP2013/056213

§ 371 Date: Sep. 23, 2014

(30) Foreign Application Priority Data
Mar. 23, 2012 (EP) 12160946.5

(51) Int. Cl. A61K 31/4184; A61K 47/32; A61K 47/34; A61K 45/06; A61K 9/00

(52) U.S. Cl. CPC A61K 31/4184; A61K 45/06; A61K 9/0053; A61K 47/34; A61K 47/32
USPC 424/1.53; 514/394; 514/47; 514/249; 514/171; 424/133.1; 514/283

(57) ABSTRACT

The present invention is related to the field of pharmaceutical compositions for the treatment of various disease states, particularly neoplastic diseases and autoimmune diseases. It especially relates to the oral application of bendamustine and its derivatives. The invention further relates to a process for preparing a pharmaceutical composition which comprises bendamustine and its derivatives prepared by hot melt extrusion using pharmaceutically acceptable excipients. The invention also relates to such pharmaceutical compositions and hot melt extrudates.

### FIELD OF THE INVENTION

The present invention is related to the field of pharmaceutical compositions for the treatment of various disease states, particularly neoplastic diseases and autoimmune diseases. It especially relates to the oral application of bendamustine and its derivatives. The invention further relates to a process for preparing a pharmaceutical composition which comprises bendamustine and its derivatives prepared by hot melt extrusion using pharmaceutically acceptable excipients. The invention also relates to such pharmaceutical compositions and hot melt extrudates.

### BACKGROUND OF THE INVENTION

Nitrogen mustards are highly reactive molecules and especially unstable in water. Thus the pharmaceutical forms of such molecules are hard to formulate and usually supplied as lyophilized form for injection which then need to be reconstituted by the hospital personal prior to injection. This step is realised with water for injection and once the molecule is dissolved in water it needs to be directly used because of the stability problems. Thus a stable oral formulation will for sure increase patient compliance as well as will avoid the problems faced during the application.

Bendamustine 4-[5-[Bis(2-chloroethyl)amino]-1-methylbenzimidazol-2-yl]butanoic acid is a nitrogen mustard containing a benzimidazole ring whose structure includes an active nitrogen mustard.

Presently, bendamustine is supplied as lyophilized powder in a brown glass due to its light instability. This lyophilized powder needs to be reconstituted prior to injection as it is the case for all nitrogen mustards. There are two different dose strengths of bendamustine, 100 mg and 25 mg. Vials are opened and 100 mg dose is reconstituted with 40 mL water for injection and 25 mg dose is reconstituted with 10 mL water for injection as close as possible to the time of administration. Furthermore these reconstituted solutions are diluted in 500 mL 0.9% sodium chloride for injection. The route of administration is by intravenous injection over 30 or 60 minutes. Once the bendamustine solution is prepared it should be given to the patient within 7 hours of vial reconstitution. Due to these stability issues, to increase the patient compliance and to overcome the difficulty due to the fact that patients are needed to be hospitalized for the administration, an oral formulation of bendamustine would be desirable.

Although bendamustine is highly orally bioavailable, there have been up to date no oral formulations of this compound. This is most probably due to high variations in oral bioavailability. In previous studies of Preis et. al. (Pharmazie 1985, 40), intravenous and oral applications of bendamustine were realized in a dose range of 4.2-5.5 mg/kg in humans. 25 mg of commercially available product Cytostasan (for intravenous applications) was filled into capsules and used as the oral formulation. As a result of pharmacokinetic studies tₘₐₓ was found to be 1 hour and mean oral bioavailability was calculated as 57% with a coefficient of variation of 44%.

In another study by Weber (Pharmazie 1991, 46:8), oral bioavailability of bendamustine was investigated in mice and found to be 40%.

The high variations in oral bioavailability of bendamustine are most probably due to the low solubility of bendamustine in pH<5.0 and low stability pH>5.0. Thus, absorption of bendamustine takes place where it is not stable resulting in high variations in bioavailability. A new oral formulation which would realise the solubility of bendamustine at low pHs and allow the absorption of this compound from early gastrointestinal tract where pH is lower than 5.0 will decrease the variations and increase the oral bioavailability allowing the oral application of this compound.

There are different patent applications available related to the oral application of bendamustine and in all of these patents solubility and stability of bendamustine is increased through excipients with conventional formulation techniques:

In patent application WO2010/063493 the formulation strategy is described as comprising bendamustine or a pharmaceutically acceptable ester, salt or solvate thereof as an active ingredient and at least one pharmaceutically acceptable excipient which compositions are said to have a good stability and an improved dissolution profile. The pharmaceutical excipient is more specifically a non-ionic surfactant which increases the solubility of bendamustine after oral application.

In patent application WO2010/126676, the oral formulation of bendamustine is realised through combination of bendamustine with one or more non-aqueous pharmaceutically acceptable excipients selected from the group of solvents and co-solvents. Specific excipients used for the formulations are either surfactant molecules alone such as PEG or in combination with other surfactant molecules. In the case of solid surfactants these were melted before the addition of bendamustine HCl. Nevertheless such formulations containing only surfactant molecules are not appropriate for hot melt extrusion due to the missing plasticizing abilities of surfactants and the brittle structure of extrudates in this case. When compared with the hot melt extrusion, by which amorphous structures can be obtained, an amorphous structure of bendamustine cannot be kept in formulations containing only surfactants. Therefore, the disadvantage appears that crystalline bendamustine still is present in the composition.

Furthermore, this document does not show that the bendamustine formulation has a sufficiently uniform and reliable bioavailability. The formulation disclosed in WO2010/126676 preferably has the form of a solid solution, solid suspension, solid dispersion, liquid dispersion, suspension, emulsion, microemulsion, gel or solution.

In patent application US20120003309, an oral formulation of bendamustine was realised with addition of pharmaceutically acceptable saccharide derivatives. Although stability of these formulations was shown, there is not sufficient information of uniform or higher bioavailability given in this application.

In patent application WO2011/151086, higher dissolution rates of the oral formulations of bendamustine were realised through incorporating surfactant excipients in the formulations. Although the coefficient of variation (CV) was decreased after the oral application there was found to be no increase in the bioavailability of the substance.

In patent application WO 2011/151087 liquid surfactants were used in order to increase the stability and bioavailability.

US2008/206350 describes a hot melt extrusion process which might include bendamustine as active ingredient, however the polymers described in the patent application are water insoluble resulting in gastric retarded formulations which will lead to degradation of bendamustine in intestines since it will not be release and absorbed in the stomach.

Pharmaceutical compositions comprising an active ingredient molecularly dispersed or dissolved in one or more polymeric carriers have been described as molecular dispersions, solid solutions or glass solutions. In general, in these types of formulations using hot melt extrusion technique, crystalline substance is in-situ dissolved in one or more polymers forming amorphous structures.

In view of the existing technologies, it is the object of the invention to provide a pharmaceutical composition comprising bendamustine suitable for oral administration. Particularly, it is the object of the present invention to provide a pharmaceutical composition comprising bendamustine for oral administration which provides a high bioavailability, low variation in oral bioavailability and increased stability. Further, it is the object of the invention to provide a pharmaceutical composition comprising bendamustine showing an increased solubility of bendamustine at a pH<5.0 as well as an increased stability at a pH of >5.0. It is an additional object of the invention to provide a pharmaceutical composition made by the HME technology comprising bendamustine having a low degree of crystallinity and a low amount of impurities.

### SUMMARY OF INVENTION

In order to achieve the above stated objects, a hot-melt extruded pharmaceutical dosage form with immediate release of bendamustine embedded in a matrix comprising at least one water soluble and/or water insoluble polymer or combination (or mixture) thereof is provided by the present invention. Preferably, the polymer is based on vinylpyrrolidon based (co)polymers.

Bendamustine is a heat sensitive substance which degrades at temperatures above 120° C. even in short term treatments. In the case of hot melt extrusion formulations using conventional polymers such as Eudragit® E100, PEG's, Soluplus and many others alone, high temperatures are required (even in the presence of plasticizers such as PEG) to be able to realise the extrusion of the polymer. In the case of lower temperatures either bendamustine crystals are present in the extrudates due to the inefficient molecular dissolution of bendamustine in the polymer system (the melting point of bendamustine is around 149-151° C.) or the characteristics of the extrudates are not appropriate for the further formulation due to the inefficient plastification. Furthermore since the complete polymer plastification is not reached the solubility of bendamustine in the polymer is not homogenous being lower at the parts, where the plastification does not occur.

Thus a new hot melt extrusion formulation, which will increase the stability of bendamustine at high temperatures and also facilitates the dissolution of bendamustine in the polymer matrix is required. Therefore, a new hot melt extrusion formulation was developed with a combination of water soluble and water insoluble polymers, preferably vinylpyrrolidon based (co)polymers such as Kollidone V64 and PVP K12 to increase the stability of bendamustine at high temperatures and to further increase the molecular solubility of bendamustine due to its hydrophilic characteristics.

Further improvements in the hot melt extrusion formulations were realised with polyoxyalkylene copolymers such as polyoxyethylene-polyoxypropylene block copolymer (for example Pluronic® F68).

Surprisingly, it turned out that the hot melt extrusion formulations of the invention were able to fully dissolve bendamustine up to at least 30 wt.% also stabilizing bendamustine at high temperatures such as 120° C. which facilitate the hot melt extrusion process. Furthermore these formulations were found to be suitable for oral administration and provide a good bioavailability and decreased coefficient of variation (CV) in bioavailability compared to prior art formulations. Furthermore the present invention discloses a method for producing hot melt extrusion formulations of bendamustine.

The inventors recognized that hot melt extrusion formulations of bendamustine increase the dissolution rate of bendamustine in the stomach and enable the absorption of this drug in early segments of the intestines offering an increased bioavailability and decreased inter-individual variability. Furthermore, the stability of bendamustine is increased through hot melt extrusion formulations making it appropriate for the oral applications.

### DETAILED DESCRIPTION OF INVENTION

As described above and further in the present application, the inventors have surprisingly found out that hot melt extrudates of bendamustine comprising a combination of water soluble and water insoluble polymers, preferably of vinylpyrrolidon based (co)polymers, increased the stability of bendamustine during the hot melt extrusion process and also increased the molecular solubility of bendamustine in the matrix formulation. Furthermore, these extrudates showed a better solubility at a pH<5.0, as well as a better bioavailability together with an increased stability at pH values >5.0.

According to a first aspect, the present invention is directed to pharmaceutical formulations comprising bendamustine or a derivative thereof as an active pharmaceutical ingredient (API) characterized in that the API is embedded in a pharmaceutically acceptable polymer matrix comprising a combination of water soluble and water insoluble polymers, preferably vinylpyrrolidon based (co)polymers. Examples of these polymers are Kollidone V64 and/or PVP K12. The polymers of the present invention allow for stable hot melt extrusion of bendamustine.

The pharmaceutical composition preferably is produced by a hot-melt extrusion process resulting in extruded products which in turn will be processed to the final pharmaceutical product such as a tablet. For example, granulates may be formed by hot-melt extrusion which may be milled subsequently and compressed (along with further excipients/auxiliaries) into tablets or may be filled into capsules.

Hot-melt extrusion (HME) is the process of transferring a powder blend of drug and carrier by a rotating screw through the heated barrel of an extruder and pressing the melt through a die into a product of uniform shape. Hot-melt extrusion thus can be simply defined as the process of forming a new material (the extrudate) by forcing it through an orifice or die under controlled conditions, such as temperature, mixing, feed-rate and pressure. Specifically, HME dosage forms are complex mixtures of API, polymers, excipients, and further auxiliaries which are blended using industry-standard equipment. The mixture is processed at elevated temperature and pressure, which disperses the drug in the matrix at a molecular level through the formation of a solid solution. The extruded material can be further processed into a variety of dosage forms, including capsules, tablets and transmucosal systems.

HME offers many advantages compared with traditional processing techniques that have been typically used to produce oral solid dosage forms. In particular, solvents are not required, which makes the process more environmentally friendly and cost effective. The end product (or extrudate) often is referred to as “solid dispersion” or “solid solution”.

The term “derivative” as used in the present application in connection with bendamustine in particular is to be understood as the pharmaceutically acceptable salt, ester or solvate form of bendamustine.

As used herein, “pharmaceutically acceptable salts” refer to derivatives of the disclosed compounds wherein the parent compound is modified by making salts thereof. For example, such conventional salts include, but are not limited to, those derived from inorganic acids such as hydrochloric, hydrobromic, sulfuric, sulfamic, phosphoric, nitric acid to name a few. Bendamustine HCl is in particular preferred.

The pharmaceutical composition of the present invention incorporates a mixture of polymers comprising at least a water soluble polymer, and at least a water insoluble polymer.

Preferred polymers according to the present invention preferably include homopolymers and co-polymers of N-vinyl lactams, especially vinylpyrrolidon based (co)polymers such as homopolymers and co-polymers of N-vinyl pyrrolidone, e.g., polyvinylpyrrolidone (PVP), co-polymers of PVP and vinyl acetate, co-polymers of N-vinyl pyrrolidone and vinyl acetate or vinyl propionate.

Further ingredients of the pharmaceutical composition of the invention include dextrins such as grades of maltodextrin, cellulose esters and cellulose ethers, high molecular polyalkylene oxides, such as polyethylene oxide and polypropylene oxide, and co-polymers of ethylene oxide and propylene oxide.

The bendamustine to polymer mixture ratio can be in between 1:0.5 to 1:100 by weight, preferably 1:1 to 1:9. The polymers are, as stated above, a mixture of water soluble and water insoluble polymers.

In a preferred embodiment, the water soluble polymer is selected from Eudragit® E100, Kollidon V64, and/or Soluplus, and where the water insoluble polymers are selected from PVP K12 and/or PEG 1000. More preferably, the polymers are Kollidon V64 and PVP K12.

The weight ratio from water soluble to water insoluble polymers preferably is about 80:20 to 90:10.

Further polymers of use in the present formulation include acrylic copolymers, e.g., Eudragit® E100 or Eudragit® EPO; Eudragit® L30D-55, Eudragit® FS30D, Eudragit® RL30D, Eudragit® RS30D, Eudragit® NE30D, Acryl-Eze® (Colorcon Co.); polyvinylacetate, for example, Kollicoat® SR 30D (BASF Co.); cellulose derivatives such as ethylcellulose, cellulose acetate, e.g., Surelease (Colorcon Co.), Aquacoat ECD and Aquacoat CPD (FMC Co.). The most preferred water soluble polymer is Eudragit® E100.

The pharmaceutical composition of the present invention additionally may contain one or more pharmaceutically acceptable excipients and/or auxiliaries.

The excipients that can be used in the further formulation of extrudates are chemically stable and nonreactive with bendamustine, or its salts, esters/solvates, under one or more of the storage conditions described herein. Excipients suitable for use in the present invention include those identified by the U.S. Food and Drug Administration as Generally Regarded as Safe (GRAS). Preferred excipients for use in the invention include solvents and co-solvents such as, for example, propylene carbonate, propylene glycol, and polyethylene glycols (for example, PEG 1000 and PEG 1500, PEG 1450, surfactants and co-surfactants such as, for example, medium chain monoglycerides such as, for example, glyceryl caprylate, glyceryl monolaurates, polyethylene glycol hydroxy stearates, polysorbates (for example, polysorbate 80), polyethylene-polypropylene glycol copolymers (for example, Poloxamer 188), tocopherol polyethylene glycol 1000 succinate, triglycerides (for example, corn oil, including super refined corn oil), and polyethylene glycol stearates, polyethylene glycol laurates, for example polyethylene glycol mono-and dilaurate mixtures, to name a few. Disintegrants, diluents, lubricants, glidants, emulsifying-solubilizing agents, sweetening agents, coating agents, antimicrobial preservatives, and the like, are also within the scope of the invention.

The amount of each excipient used within the scope of the invention will vary, depending on the particular excipients selected. Preferably, the pharmaceutical compositions of the invention will include at least one pharmaceutically acceptable excipient.

Applicable solid excipients can include one or more substances that may also act as flavoring agents, lubricants, solubilizers, suspending agents, fillers, glidants, compression aids, binders or tablet-disintegrating agents or an encapsulating material. In powders, the excipient is a finely divided solid that is in admixture with the finely divided active ingredient, i.e. bendamustine or a pharmaceutically acceptable salt thereof. In tablets, the active ingredient is mixed with an excipient having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Suitable drug dosage forms include, but are not limited to, tablets, for example, immediate-, controlled-, and extended-release tablets, pills, capsules, soft gels, sachets, granules, powders, chewing gums, solid suspensions, emulsions, and solutions.

Liquid excipients may be used in preparing solutions, suspensions, emulsions, syrups, and elixirs. Extrudates comprising bendamustine or a pharmaceutically acceptable salt, ester or solvate thereof, can be suspended in a pharmaceutically acceptable liquid excipient such as an organic solvent or pharmaceutically acceptable oils or fat. The liquid excipient can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid excipients for oral administration alcohols (including monohydric alcohols and polyhydric alcohols e.g. glycols) and their derivatives, oils (e.g. fractionated coconut oil and arachis oil), and for short contact periods, water (particularly containing additives as above, e.g. cellulose derivatives, preferably sodium carboxymethyl cellulose solution).

Examples of auxiliaries are plasticizers which can be used in order to lower the glass transition of the polymers include, but are not limited to, polysorbates such as sorbitan monolaurate (Span 20), sorbitan monopalmitate, sorbitan monostearate, sorbitan monoisostearate; citrate ester type plasticizers like triethyl citrate, citrate phthalate; propylene glycol; glycerin; polyethylene glycol (low and high molecular weight); triacetin; dibutyl sebacate, tributyl sebacate; dibutyltartrate, dibutyl phthalate. Preferably, polyoxyalkylene copolymers such as polyoxyethylene-polyoxypropylene block copolymer (for example Pluronic® F68) are used as plastizisers. The plasticizers are generally included in the formulation in about 10% of the polymer amount.

Furthermore antioxidants and other stabilizing agents can be included in the formulations. These substances include but not limited to ascorbic acid, butylhydroxyanisole (BHA), butylhydroxytoluene (BHT), salts of ascorbic acid, monothioglycerol, phosphorous acid, vitamin C, vitamin E and the derivatives thereof, coniferyl benzoate, nordihydroguajaretic acid, gallus acid esters, sodium bisulfite, particularly preferably butylhydroxytoluene or butylhydroxyanisole and α-tocopherol.

In an embodiment, the pharmaceutical composition of the invention contains one or more additional active pharmaceutical ingredients (API's) and/or pharmaceutically acceptable auxiliaries. Those API's may be preferably selected from etoposide, fludarabine, mitoxantrone, methotrexate, prednisone, rituximab, vincristine and/or 90Y-ibritumomab tiuxetan.

The extrudates formed by hot-melt extrusion can be designed to be used in a variety of different pharmaceutical compositions and formulations such as oral delivery as tablets, capsules or suspensions, pulmonary and nasal delivery, topical delivery as emulsions, ointments and creams, and parenteral delivery as suspensions, microemulsions or as a depot. Oral delivery is most preferred.

Preferably the pharmaceutical composition is in single unit dosage form, e.g. as tablets, capsules, powders, solutions, suspensions, emulsions, or granules. In such form, the composition is sub-divided in unit dose containing appropriate quantities of the active ingredient; the unit dosage forms can be packaged compositions, for example packaged powders, vials, ampoules, and the like. The unit dosage form can be, for example, a capsule or tablet itself, or it can be the appropriate number of any such compositions in package form.

The daily dosage of bendamustine might be, but is not limited to the range of 50 to about 1000 mg/day and most preferably 100 to about 500 mg of the active ingredient. A single dose may vary between 25 mg and 100 mg of bendamustine.

A further aspect of the present invention is the use of the pharmaceutical composition of the present invention in the treatment of neoplastic or autoimmune diseases. Neoplastic diseases, which may be treated comprise chronic lymphocytic leukemia, Hodgkin's disease, non-Hodgkin's lymphoma, multiple myeloma and lung cancer.

According to a further aspect, the pharmaceutical composition of the present invention may be prepared through hot melt extrusion technique which involves hot melt extrusion of bendamustine with a mixture of one or more water soluble and one or more water insoluble polymers using a conventional extruder as known to a person skilled in the art.

As such, the method of producing a pharmaceutical composition comprising bendamustine or a derivative thereof as an API comprises the steps of:
mixing and melting the API and a mixture of water soluble and water insoluble polymers in order to provide a melt,
extruding the melt through a die thereby forming an extrudate; and
cooling the extrudate until it solidifies.

In general, hot melt extrusion technique includes steps of preparing a homogenous melt of bendamustine or its pharmaceutically approved derivatives, the polymer and the excipients followed by cooling the melt until it solidifies. Typically one component will melt dissolving the other components and forming a solid solution. Mixing of the components can take place before, after or during the melting step.

Usually the melt temperature is in the range of about 50° C. to 200° C. However, according to the invention, it is preferred to use temperatures up to and including about 120° C. and thus a more preferred temperature range will include 80-140° C. If the temperature is higher, degradation of bendamustine might occur. A most preferred range is from 90-130° C. As outlined above, a temperature up to about 120° C. still is sufficient to dissolve bendamustine under the proviso that the polymer mixture of the present invention is used as a polymer matrix material. Under these conditions, crystallization of bendamustine may be avoided.

Different extruders can be used for this process for example but not limited to single screw extruders or multiscrew extruders. Extrudates can have different shapes such as but not limited to rhodes, granulates, tubes or beads and can be further processed into any desired shape as outlined above.

In a further aspect, the present invention discloses a pharmaceutical composition obtainable by the above method and its oral, topical, pulmonary, nasal, or parenteral use. Furthermore, the pharmaceutical composition of the invention may be used in the treatment of neoplastic or autoimmune diseases.

The present application now is described in more detail by the following Examples. However, it is noted that the Examples are provided for illustrative purposes only and should not be construed to limit the scope of the invention in any way.

### EXAMPLES

### Example 1

Compatibility studies of bendamustine with different excipients used in hot melt extrusion process were realised with incubation of 1:1 (w/w) binary mixtures of these excipients with bendamustine at 40° C. 75% RH in an open container for one month.

TABLE 1
Results of compatibility analyses of bendamustine with different excipients

| | | | | Ditert butyl | | | | | | |
|---|---|---|---|---|---|---|---|---|---|---|
| | Kollidone | PEG | PEG | methyl | Ascorbic | Plasdone | Citric | Vit E | Solu- | Eudragit ® |
| | VA64 | 2000 | 1000 | phenol | acid | K12 | acid | acetate | plus | EPO |
| Total | 0.091 | 0.229 | 0.326 | 0.258 | 0.121 | 0.124 | 0.098 | 0.101 | 0.118 | 0.154 |
| impurities | | | | | | | | | | |
| % | | | | | | | | | | |

According to the total impurities of Bendamustine after incubation at 40° C. 75% RH together with the excipients, incompatibility of bendamustine with large amounts of PEG 2000, PEG 1000 and Diter butyl methyl phenol was observed. Further, it turned out that the use of vinylpyrrolidon-based polymers such as Kollidone resulted in a very low amount of impurities.

### Example 2

Temperature stability studies of bendamustine were conducted with treatment of bendamustine at given temperatures for 10 minutes.

TABLE 2
Assay and total impurity results of temperature
stability studies of bendamustine

| Temperature ° C. | Total Impurity % | Assay % |
|---|---|---|
| 70 | 0.7 | 101.75 |
| 100 | 0.8 | 99.216 |
| 120 | 1.3 | 97.732 |

According to the results in Tab. 2 temperatures higher than 100° C. resulted in the increased degradation of bendamustine.

### Example 3

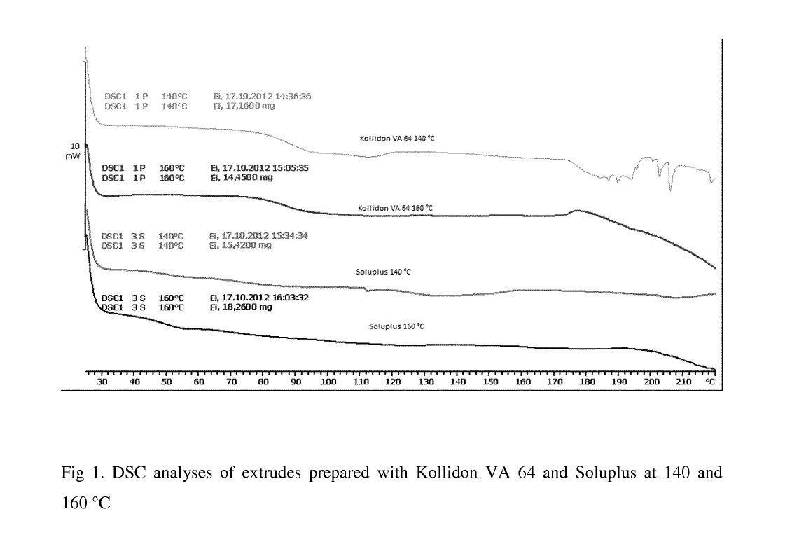

Preparation of bendamustine (10%) extrudates using Soluplus (70%) or Kollidon VA 64 (70%) with addition of Pluronic (20%) as plasticizer were realised at 140° C. and 160° C. using a twin screw extruder at a speed of 50 rpm. DSC analyses were conducted to determine the morphology of bendamustine (FIG. 1) and additionally impurity analyses were conducted to determine the impurity profiles.

TABLE 3
Impurity profiles of extrudates using Soluplus and
Kollidon VA 64 at two different temperatures

| Excipient | Temperature ° C. | Total impurity % |
|---|---|---|
| Kollidon VA 64 | 140 | 2.30 |
| Kollidone VA 64 | 160 | 3.53 |
| Soluplus | 140 | 1.45 |
| Soluplus | 160 | 2.00 |
| Eudragit ® E100 | 140 | 3.5 |
| Eudragit ® E100 | 160 | 4.2 |

Although DSC analyses shows the amorphous structure of bendamustine in all samples, extrusion of bendamustine at 140° C. and 160° C. regardless of the polymer type used resulted in high total impurity levels showing the degradation of bendamustine during the extrusion process.

### Example 4

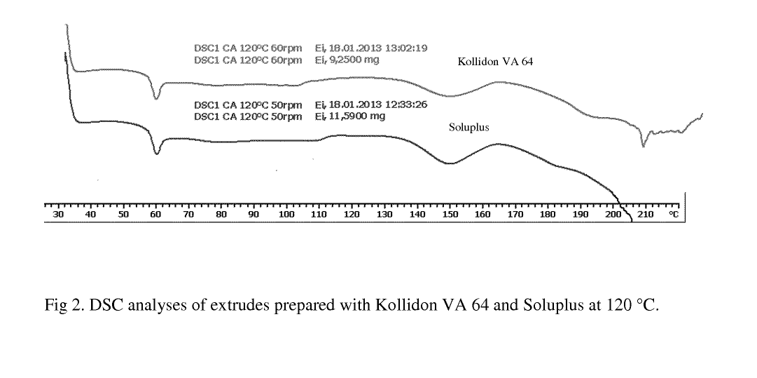

Hot melt extrusion studies with Kollidon VA 64 and Soluplus were conducted as described in Example 3 but at 120° C. Furthermore a combination of Kollidon VA 64 with beta cyclodextrin was extruded at 120° C. using a twin screw extruder at a speed of 50 rpm. DSC analyses were conducted to determine the morphology of bendamustine (FIG. 2) and additionally impurity analyses were conducted to determine the impurity profiles.

TABLE 4
Impurity profiles of extrudes using Soluplus
and Kollidon VA 64 at 120° C.

| Excipient | Temperature ° C. | Total impurity % |
|---|---|---|
| Kollidon VA 64 | 120 | 0.821 |
| Soluplus | 120 | 0.951 |
| Kollidon VA 64/cyclodextrin | 120 | 1.201 |

Further decrease in the temperature to maintain the stability of bendamustine during the extrusion process resulted in lowered total impurity levels but as shown with DSC analyses regardless of the polymer used there was crystalline bendamustine present in all samples. Thus the extrusion process was not complete and bendamustine was not fully dissolved in the polymers used.

### Example 5

Different combinations of water soluble and water insoluble polymers were tested in order to stabilize bendamustine during extrusion and to be able to fully dissolve bendamustine at lower temperatures. Thus extrusion experiments were carried out 120° C. using a twin screw extruder at a speed of 50 rpm. Eudragit° E100, Kollidon V64, Soluplus were used as water insoluble polymers and PVP K12 and PEG 1000 were used as water soluble polymers.

TABLE 5
Composition of extrudates

| | F1 | F2 | F3 | F4 |
|---|---|---|---|---|
| Bendamustine | 20% | 20% | 20% | 20% |
| Eudragit ® E 100 | — | 63% | — | — |
| Kollidon V64 | 70% | — | 63% | — |
| Soluplus | — | — | — | 60% |
| Pluronic F68 | — | — | — | 10% |
| PVP K12 | — | — | — | 10% |
| PEG 1000 | 10% | 10% | 10% | — |
| Butylhydroxytoluene | — | 2% | 2% | — |
| Ascorbic acid | — | 5% | 5% | — |

With the combination of water soluble and insoluble polymers, it was possible to inhibit the degradation of Bendamustine during the extrusion process. The impurities were found to be 0.32, 0.28, 0.30, 0.20 for F1, F2, F3, F4 respectively. Furthermore DSC analyses showed that bendamustine was completely dissolved in polymer matrix.

### Example 6

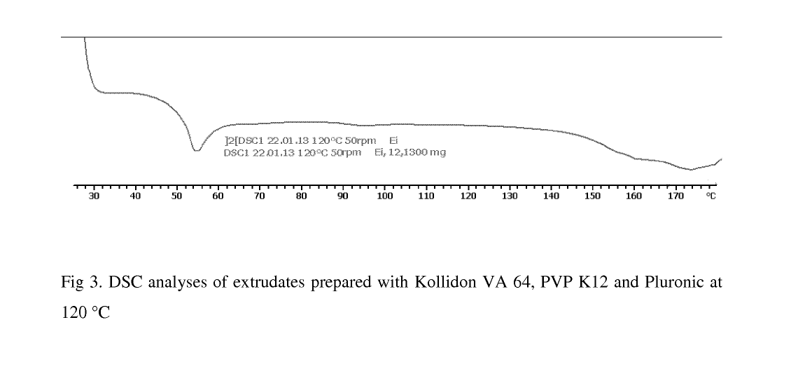

Hot melt extrusion studies were conducted using Kollidone VA 64, PVP K12 and Pluronic at 120° C. using a twin screw extruder at a speed of 50 rpm. DSC analyses were conducted to determine the morphology of bendamustine (FIG. 3) and additionally impurity analyses were conducted to determine the impurity profiles. Total impurities were found 0.14%. Using the formulation shown in Tab. 5 bendamustine was fully dissolved in polymer composition as shown with the DSC analyses and furthermore stability of bendamustine during the extrusion was increased even at 120° C. resulting in low impurity profiles.

TABLE 6
Composition of extrudates

| Ingredient | (wt.-%) |
|---|---|
| Bendamustine | 30 |
| Kollidon V64 | 30 |
| Pluronic F68 | 15 |
| PVP K12 | 20 |
| Ascorbic acid | 5 |

### Example 7

Capsule formulations were prepared from the extrudates gathered from Example 5 as follows

| | |
|---|---|
| Bendamustine extrudate | 61.5 wt.-% |
| Avicel pH 102 | 29.3 wt.-% |
| Crospovidone | 9.2 wt.-% |

The above stated mixture was filled in size 0 capsules.

### Example 8

Dissolution studies were conducted with extrudates filled in hard gelatine capsules and 100% dissolution was observed in less than 20 minutes as measured with paddle apparatus at 75 rpm according to the European Pharmacopoeia in 500 mL of a dissolution medium at a pH of 1.2.

TABLE 7
Dissolution of capsules

| | 10 min | 20 min | 30 min | 45 min |
|---|---|---|---|---|
| % Dissolution | 77.51 | 96.12 | 95.91 | 95.10 |

### Example 9

Assay and impurity analyses of extrudates were conducted with HLPC-UV methods validated in accordance with cGMP regulations.

TABLE 8
Stability of extrudates at 25° C. 60% RH

| Assay | Assay | Total impurity | Total impurity |
|---|---|---|---|
| 0. month [%] | 1. month [%] | 0. month [%] | 1. month [%] |
| 99.5 | 99.3 | 0.14 | 0.17 |

TABLE 9
Stability of extrudates at 40° C. 75% RH

| Assay | Assay | Total impurity | Total impurity |
|---|---|---|---|
| 0. month | 1. month | 0. month | 1. month |
| 99.5 | 99.2 | 0.14 | 0.19 |

### Example 10

Rat studies were carried out with the extrudate to determine the oral bioavailability of bendamustine. For this purpose 3 mg/kg bendamustine was applied parenterally to 3 animals and samples were taken at 0, 0.083, 0.25, 0.5, 1, 2, 4, 8 hours and analyzed with a validated HPLC/MS method. Furthermore extrudates of the invention were applied orally to 9 rats with a bendamustine concentration of 10.8 mg/kg. Samples were taken at 0, 0.25, 0.5, 1, 2, 4, 6, 8 hours and analyzed with a validated HPLC/MS method

| Animal Species | rat, male, Sprague-Dawley | | |
|---|---|---|---|
| Body weight range (g) | 279-344 | | |
| Number of animals | 3 | 3 | 9 |
| Test item | Bendamustine | Bendamustine | Extrudate |
| Dose route | iv | po | po |
| Dosage (mg/kg) | 3 | 10 | 10.8 |
| mean AUC0-∞(ng*h/ml) | 675 | 742.5 | 1601 |
| F(%) | 100% | 33% | 66% |
| CV % | — | — | 27% |

As it can be seen at the table above there is a 2 fold increase in the oral bioavailability of bendamustine when extrudates are applied orally. Furthermore the CV % was found to be 27%.

Bendamustine is a heat sensitive substance which negatively affects its potential to be good a candidate for hot melt extrusion. As described in Tab. 2, temperatures higher than 100° C. results in degradation of bendamustine. However, such temperatures are needed for the hot melt extrusion process for the plastification of pharmaceutical polymers used in this process. As described in example 3, when bendamustine is extruded at high temperatures such as 140° C. and 160° C.—although it is completely dissolved in the polymers as shown in FIG. 1—the total impurity profiles increased up to 3.58% which is higher than acceptable limits independent of the polymer used for extrusion. Furthermore decrease in temperature down to 120° C. resulted in decrease in the impurity profiles but as shown in FIG. 2 results in crystalline bendamustine in extrudates even at a concentration of 10 wt.-% regardless of the polymer used. Furthermore extrudates gathered in such low temperatures are brittle and not appropriate for further formulation studies.

Thus new formulations were developed. Surprisingly it was found out that the use a mixture of water soluble and water insoluble polymers, preferably of vinylpyrrolidon based (co)polymers results in fully amorphous bendamustine with a total impurity of 0.14% at 120° C. indicating that these polymers increased the stability of bendamustine during hot melt extrusion process. Further dissolution studies revealed an immediate release from extrudates which will result in immediate absorption of bendamustine and decrease in the CV %. Using the developed formulation bendamustine content of extrudates can be increase up to 30% which was not possible due to crystallization of bendamustine using the conventional hot melt extrusion formulations.

Thus, a formulation of the invention suitable for hot melt extrusion of bendamustine or a pharmaceutically acceptable salt inhibits the degradation of bendamustine or a pharmaceutically acceptable salt during the extrusion process.

Furthermore, in such a hot melt extrusion formulation bendamustine or a pharmaceutically acceptable salt can be completely dissolved at low temperatures such as 120° C. resulting in amorphous bendamustine or a pharmaceutically acceptable salt thereof.

The present invention further provides a hot melt extrusion formulation of bendamustine or a pharmaceutically acceptable salt which is stable throughout the storage time with a higher bioavailability and a reduced CV %

1. A pharmaceutical composition comprising bendamustine or a derivative thereof as an active pharmaceutical ingredient (API), wherein the API is embedded in a pharmaceutically acceptable polymer matrix comprising a mixture of one or more water soluble polymers and one or more water insoluble polymers.

2. The pharmaceutical composition of claim 1, wherein the one or more water soluble polymers and the one or more water insoluble polymers are vinylpyrrolidone based (co)polymers.

3. The pharmaceutical composition of claim 1, wherein the one or more water soluble polymers are selected from the group consisting of EUDRAGIT® E100, KOLLIDON® V64, and/or SOLUPLUS®, and where the water insoluble polymers are selected from the group consisting of polyvinylpyrrolidone (PVP) K12, and PEG 1000.

4. The pharmaceutical composition of claim 2, wherein the water soluble polymer is KOLLIDON® V64 and the water insoluble polymer is PVP K12.

5. The pharmaceutical composition of claim 1, wherein the API is finely dispersed or dissolved in the polymer matrix.

6. The pharmaceutical composition claim 1, wherein pharmaceutical composition has a weight ratio-of the API to total polymer of 1:0.5 to 1:100.

7. The pharmaceutical composition of claim 1, wherein the total water soluble polymer to total water insoluble polymer are present in the pharmaceutical composition in a weight ration of about 80:20 to 90:10.

8. The pharmaceutical composition of claim 1, wherein the pharmaceutical composition further comprises one or more additional active pharmaceutical ingredients (APIs), pharmaceutically acceptable excipients, auxiliaries, or a combination thereof.

9. The pharmaceutical composition of claim 8, wherein the one or more additional APIs are selected from the group consisting of etoposide, fludarabine, mitoxantrone, methotrexate, prednisone, rituximab, vincristine and 90Y-ibritumomab tiuxetan.

10. A method of producing a pharmaceutical composition comprising bendamustine or a derivative thereof as an active pharmaceutical ingredient (API), the method comprising:
mixing and melting the API and a mixture of one or more water soluble polymers and one or more water insoluble polymers in order to provide a melt,
extruding the melt through a die thereby forming an extrudate; and
cooling the extrudate until it solidifies.

11. The method according to claim 10, wherein the API is admixed with the polymer before, during or after melting said polymer.

12. The method according to claim 11, wherein at least one pharmaceutically acceptable excipient or auxiliary is dispersed or dissolved in the polymer.

13. The method of claim 10, wherein the melt is formed at a temperature between 50° C. and 200° C.

14. A pharmaceutical composition produced by the method of claim 10.

15. The pharmaceutical composition of claim 14 formulated for oral, topical, pulmonary, nasal, or parenteral use.

16. The pharmaceutical composition of claim 14 for use in the treatment of neoplastic or autoimmune diseases.

17. The method of claim 13, wherein the melt is formed at a temperature between 80° C. and 140° C.

18. The method of claim 17, wherein the melt is formed at a temperature between about 90° C. and 130° C.
